(19) United States
(12) Patent Application Publication
(10) Pub. No.: US 2024/0329746 A1
(43) Pub. Date: Oct. 3, 2024

(54) METHOD FOR GUIDING USAGE OF MOBILE SCREEN

(71) Applicant: Dan GONEN, Kiryat Ono (IL)

(72) Inventor: Dan GONEN, Kiryat Ono (IL)

(21) Appl. No.: 18/198,311

(22) Filed: May 17, 2023

(30) Foreign Application Priority Data
Mar. 29, 2023 (IL) 301778

(51) Int. Cl. G06F 3/01

(52) U.S. Cl. CPC G06F 3/017; G06F 3/012

(57) ABSTRACT

A method for guiding usage of a mobile screen, the method including the steps of: while a user uses the mobile screen: measuring tilt of the mobile screen; and awarding the user a score, based on the tilt measurement, the first score improving for minimizing the tilt.

### CROSS-REFERENCE TO RELATED APPLICATIONS

This application claims the benefit of priority from Israel Patent Application No. 301778, filed Mar. 29, 2023, the disclosure of which is incorporated herein by reference.

### TECHNICAL FIELD

The invention relates to the field of mobile screen applications. More particularly, the invention relates to a method for guiding usage of a mobile screen.

### BACKGROUND

While using a mobile screen, the user's head tilts spontaneously without feeling downwards. Young and old people use, read, write, and play for many hours every day on mobile screens. Over time, the position of their head downwards during use becomes a permanent habit, this position can cause scoliosis in the neck, spine, etc. During negative health effects, the user is immersed in the contents of the device and does not notice that his head is in an unhealthy and unsightly state.

There is a long felt need to provide a solution to the above-mentioned and other problems of the prior art.

### SUMMARY

The invention, in embodiments thereof, provides a method for guiding usage of a mobile screen, the method including the steps of, while a user uses the mobile screen, measuring tilt of the mobile screen, and awarding the user a score, based on the tilt measurement, the score improving for minimizing the tilt (16A).

### BRIEF DESCRIPTION OF THE DRAWINGS

Embodiments, features, and aspects of the invention are described herein in conjunction with the following drawings:

FIG. 1 is a front view of a user holding a mobile screen desirably, and the measurement thereof.

FIG. 2 is a front view of a user holding a mobile screen differently from the position of FIG. 1.

FIG. 3 is a front view of a user holding a mobile screen differently from the position of FIG. 1.

FIG. 4 is a front view of a user holding a mobile screen differently from the position of FIG. 1.

The drawings are not necessarily drawn to scale.

### DETAILED DESCRIPTION

The invention will be understood from the following detailed description of embodiments of the invention, which are meant to be descriptive and not limiting. For the sake of brevity, some well-known features are not described in detail.

The reference numbers have been used to point out elements in the embodiments described and illustrated herein, in order to facilitate the understanding of the invention. They are meant to be merely illustrative, and not limiting. Also, the foregoing embodiments of the invention have been described and illustrated in conjunction with systems and methods thereof, which are meant to be merely illustrative, and not limiting.

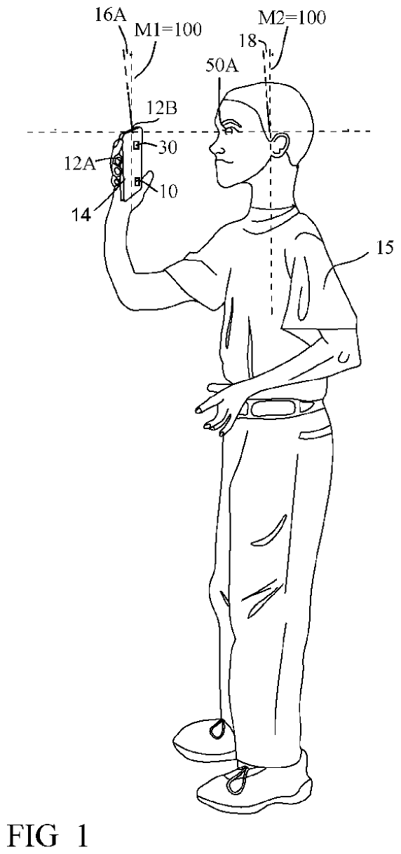

FIG. 1 is a front view of a user holding a mobile screen desirably, and the measurement thereof.

An application 10 running on a mobile screen 14, together with any other application 30 being currently run by the user 15, utilizes the tilt sensor 12A, being the inertial measurement unit (IMU) of mobile screen 14, for measuring the angle 16A of the shift of mobile screen 14.

In the case of FIG. 1 angle 16A is 0 degrees, being the desirable angle, thus giving a good score M1 for the angle, say 100.

Application 10 running on a mobile screen 14 further utilizes the camera 12B of mobile screen 14, for capturing the user's eye 50A.

In the case of FIG. 1 camera 12B is located at the height of eye 50A, being the desirable height, and thus captures eye 50A, thus giving a good score M2 for the height, say 100.

The total score of M1=100 and M2=100 is thus 100. This score is correct since the user's head is not tilted at all, as shown by angle 18 of the user's head being 0 degrees.

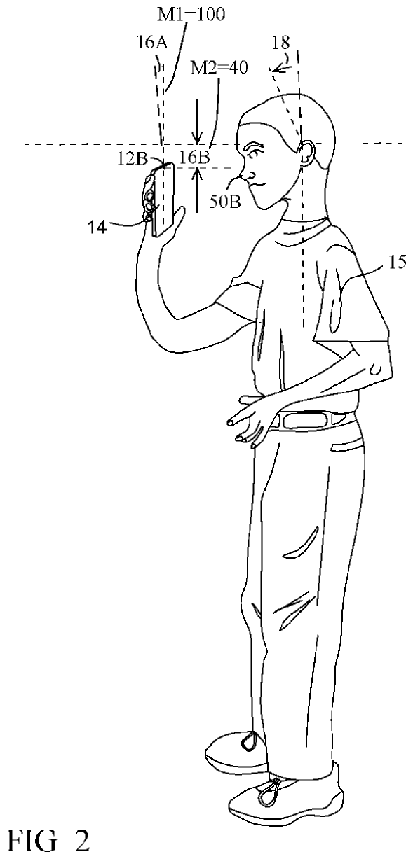

FIG. 2 is a front view of a user holding a mobile screen differently from the position of FIG. 1.

For the holding of FIG. 2 angle 16A, as measured by accelerometer 12A, as well is 90 degrees, being the desirable angle, thus giving a good score M1 for the angle, say 100.

However, for the holding of FIG. 2, camera 12B as captured thereby, is located at the height of nose 50B, being below the desirable height, and thus captures nose 50B, thus giving a bad score M2 for the height, say 60.

The total score of M1=100 and M2=60 is thus 80. This score is correct since angle 18 of the user's head is 30 degrees.

A low score may produce an alarm to the user, and may share the information with groups of users, and may suggest gym for responding to the scores of this and other users.

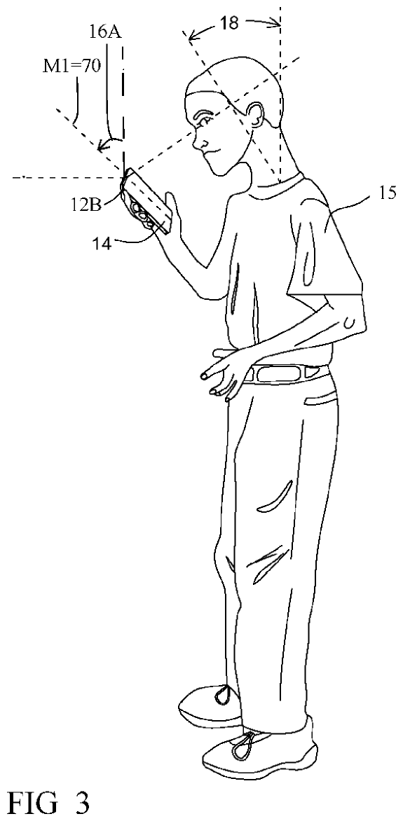

FIG. 3 is a front view of a user holding a mobile screen differently from the position of FIG. 1.

For the holding of FIG. 3 angle 16A, as measured by accelerometer 12A, is 45 degrees, being a non-desirable angle, thus giving a bad score M1 for the angle, say 50.

However, for the holding of FIG. 3, camera 12B is located at the height of eye 50A, being the desirable height, and thus captures eye 50A, thus giving a good score M2 for the height, say 100.

The total score of M1=50 and M2=100 is thus 75. This score is correct since angle 18 of the user's head is 30 degrees.

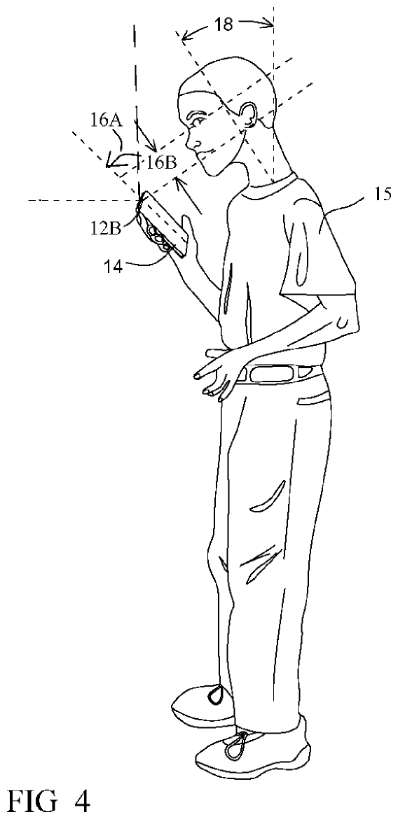

FIG. 4 is a front view of a user holding a mobile screen differently from the position of FIG. 1.

For the holding of FIG. 4 angle 16A, as measured by accelerometer 12A, is 45 degrees, being a non-desirable angle, thus giving a bad score M1 for the angle, say 50.

Further, for the holding of FIG. 4, camera 12 is located at the height of nose 50B, being below the desirable height, and thus captures nose 50B, thus giving a bad score M2 for the height, say 60.

The total score of M1=50 and M2=60 is thus 30. This score is correct since angle 18 of the user's head is greater than 45 degrees.

Thus, in one aspect, the invention is directed to a method for guiding usage of a mobile screen (14), the method including the steps of: while a user (15) uses the mobile screen (14) measuring tilt (16A) of the mobile screen (14), and awarding the user a first score (M1), based on the tilt measurement, the first score improving for minimizing the tilt (16A).

The tilt measuring and the score awarding are operated by an application (10) run by the mobile screen (14).

The steps of tilt measuring and of the score awarding may be repeated over time, thereby calculating an average score.

The tilt measuring may be applied by a tilt sensor (12A) of the mobile screen.

The method may further include the steps of measuring shift (16B) of the mobile screen in relation to the face (50A, 50B) of the user, and awarding the user a second score (M2), based on the shift (16B), the second score improving for minimizing the shift (16B).

The shift measuring may be applied by a front camera (12A) of the mobile screen.

The method may further include the step of awarding the user a third score, the third score including a combination of the first (M1) and second (M2) scores.

The method may further include the step of calculating tilt (18) of a head of the user, the calculation based on at least one of the first (M1) and second (M2) scores.

In the figures and description herein, the following reference numbers (Reference Signs List) have been mentioned:
number 10 denotes the application for guiding the user, according to one embodiment of the invention;
M1, M2: scores;
12A: tilt sensor of mobile screen 14;
12B: camera of mobile screen 14;
14: mobile screen;
15: user;
16A: measured tilt of mobile screen 14;
16B: measured shift of mobile screen 14 in relation to the user's face;
18: tilt of the user's head, not being directly measured, but rather being calculated from tilt 16A and shift 16B;
30: prior art application being currently run by the user;
50A: user's eye;
50B: user's nose.

The foregoing description and illustrations of the embodiments of the invention have been presented for the purpose of illustration and are not intended to be exhaustive or to limit the invention to the above description in any form.

Any term that has been defined above and used in the claims, should be interpreted according to this definition. The reference numbers in the claims are not a part of the claims, but rather used for facilitating the reading thereof. These reference numbers should not be interpreted as limiting the claims in any form.

1. A method for guiding usage of a mobile screen, the method comprising the steps of:
while a user uses said mobile screen:
measuring, by a tilt sensor of said mobile screen, tilt position of said mobile screen;
calculating tilt position of a head of the user, based on said measured tilt position, and based on assuming that the user is looking at said mobile screen;
awarding the user a first score, based on said calculating of said tilt position of the user's head, said first score increasing for minimizing said tilt position of the user's head; and
providing an alarm to the user once said first score is low.

2. The method according to claim 1, wherein said tilt measuring and said score awarding are operated by an application run by said mobile screen.

3. The method according to claim 1, wherein said steps of tilt measuring and of said score awarding are repeated over time, thereby calculating an average score.

4. (canceled)

5. The method according to claim 1, further comprising the steps of:
measuring shift of said mobile screen in relation to a face of the user; and
awarding the user a second score, based on said shift, said second score improving for minimizing said shift.

6. The method according to claim 5, wherein said shift measuring is operated by a front camera of said mobile screen.

7. The method according to claim 5, further comprising the step of:
awarding the user a third score, said third score comprising a combination of said first and second scores.

8. (canceled)
